(19) United States
(12) Patent Application Publication
(10) Pub. No.: US 2010/0168571 A1
(43) Pub. Date: Jul. 1, 2010

(54) IMAGE-BASED POWER FEEDBACK FOR OPTIMAL ULTRASOUND IMAGING OR RADIO FREQUENCY TISSUE ABLATION

(75) Inventors: David Savery, Calries (FR); Christopher Hall, Hopewell Junction, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/377,060

(22) PCT Filed: Aug. 2, 2007
(86) PCT No.: PCT/IB07/53047

§ 371 Date: Feb. 10, 2009

Related U.S. Application Data
(60) Provisional application No. 60/822,125, filed on Aug. 11, 2006.

(51) Int. Cl. A61B 8/14; A61B 18/18

(52) U.S. Cl. 600/439; 606/33

(57) ABSTRACT

Methods and systems are provided for monitoring and regulating radiofrequency (RF) ablation therapy to improve quality of ultrasound imaging. Feedback is provided from real-time ultrasound imaging, and RF power is altered in response to a feedback signal to improve image quality.

The technical field of the invention is providing a method and system for optimizing ultrasound images during radiofrequency (RF) ablation by providing feedback from real-time imaging and controlling RF power.

RF ablation is a curative, clinical procedure often used for tumor destruction in treating diverse classes of cancer, such as hepatic metastases or hepatocellular carcinoma. RF ablation is a promising procedure for treating cancer patients who cannot undergo resection surgery. The clinical objective of RF ablation is to thermally ablate cancerous tissue while sparing healthy parenchyma to ensure that side effects of treatment are minimal.

RF ablation is a minimally invasive procedure that needs to be guided and monitored by an external interventional imaging modality. Currently, imaging modalities that are most commonly used for guidance and monitoring of RF ablation are ultrasound and computed tomography. As ultrasound scans provide real-time images, with virtually no harmful radiation and at a relatively moderate cost, this technique has great existing and untapped promise for guidance and monitoring of interventional procedures. Advantages of ultrasound include its real time capabilities and cost aspects, however due negative impacts of cavitation resulting from intensity of heating during RF treatment, ultrasound image quality can be diminished.

During RF ablation treatment, body temperature is increased locally at levels to induce necrosis, i.e. death of cells or tissue, in a targeted area. An RF probe is inserted into the target tissue, usually percutaneously. Heat is produced by dielectric loss, at the passage of a RF current generated at the tip(s) of the probe. During heating, the temperature of the tissue surrounding the tip of the probe can reach the boiling point (close to 90-100° C.), which results in cavitation, i.e. the formation of bubble pockets. The presence of bubbles affects the propagation of an acoustic imaging wave through the medium, and disrupts ultrasound image quality. When bubbles are present, the efficacy of ultrasonic monitoring of the procedure is readily degraded by the “shadowing” or loss of signal distal to a gas pocket. Furthermore, generation of bubbles can also modify the outcome of the treatment itself, since air is a good insulator and can therefore prevent heat diffusion within tissue. It is therefore desirable to prevent bubble formation to improve visualization of the RF ablation procedure on the ultrasound scanner.

Accordingly, an object of the present invention is optimizing ultrasound images by controlling RF power to minimize the formation of bubbles, while at the same maximizing the efficacy of RF ablation therapy. Using the ultrasound data supplied by an ultrasound imaging system as feedback parameters, the RF power generated during RF ablation treatment is limited in order to avoid heat-induced cavitation.

A featured embodiment of the invention provided herein is a method for monitoring and regulating radiofrequency (RF) ablation therapy to improve quality of imaging, the method including: imaging a target area using an ultrasound imaging system to provide a pre-treatment image for calibration, and maintaining continuous real-time acquisition of at least one additional image; inserting an RF probe into the target area and generating an RF current to heat the target area near a tip of the RF probe, and producing at least one intra-operative image from the continuous real-time acquisition; and comparing the pre-treatment image and the intra-operative image to generate a feedback signal, wherein the feedback signal is relayed to an RF power generator, and altering RF power in response to the feedback signal, to improve quality of the intra-operative image.

In a related embodiment, the method includes comparing the pre-treatment image and the intra-operative image, further responding to an index that determines a presence in the target area of at least one bubble. The index is derived from an ultrasonic image that indicates the presence of bubbles. As bubbles often appear as highly echogenic pockets, a decision can be made on the examination of several ultrasound features. For example, the feedback signal includes a variation in an acoustic feature. In a related embodiment, the acoustic feature is at least one of: a variation in echogenicity, a variation in Doppler spectra in duplex imaging, and a non-linear detection scheme. Further, the non-linear detection scheme comprises harmonic signals and/or sub-harmonic signals.

In another related embodiment, comparing the pre-treatment image and the intra-operative image further involves obtaining a thermocouple reading or an impedance reading.

Another featured embodiment of the invention herein is a system that includes: an ultrasound scanner that acquires a pre-treatment image of a target area for calibration, and at least one additional image of the target area; a radiofrequency (RF) probe, such that the RF probe is inserted into the target area; an RF power generator; and a bubble detector, such that the bubble detector indicates a presence of at least one bubble in the target area and produces a feedback signal, and such that the RF power generator is altered in response to the feedback signal.

In a related embodiment, the bubble detector further compares the pre-treatment image and at least one intra-operative image. In an alternative embodiment, the bubble detector includes at least one of: a passive cavitation detector, a microphone, and a stethoscope. For example, the bubble detector determines a variation in echogenicity, a variation in Doppler spectra in duplex imaging, and a non-linear detection scheme. Further, the non-linear detection scheme includes harmonic signals and sub-harmonic signals.

In a related embodiment, detection of a presence in the target area of at least one bubble initiates at least one event in a closed loop feedback system. For example, the event includes an alteration in RF power. For example, the alteration in RF power includes an alteration of power to at least one tip of the RF probe. Further, the event includes a temporary extinction of an RF power generator signal.

In an alternative or an additional embodiment, a user is notified of a detection of a presence in the target area of at least one bubble, and the user initiates at least one event in an open loop feedback system. For example, the event includes an alteration of RF power. Further, the alteration in RF power includes an alteration of power to at least one tip of the RF probe. Further, the event includes a temporary extinction of an RF power generator signal.

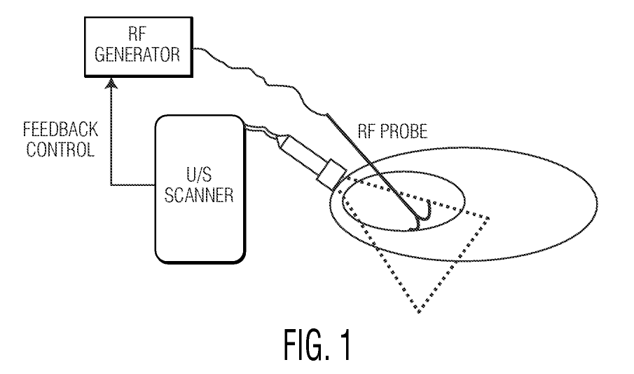

FIG. 1 is a diagram showing an ultrasound scanner, a RF probe or electrode, and an RF power generator, with the ultrasound scanner providing feedback control to the RF power generator.

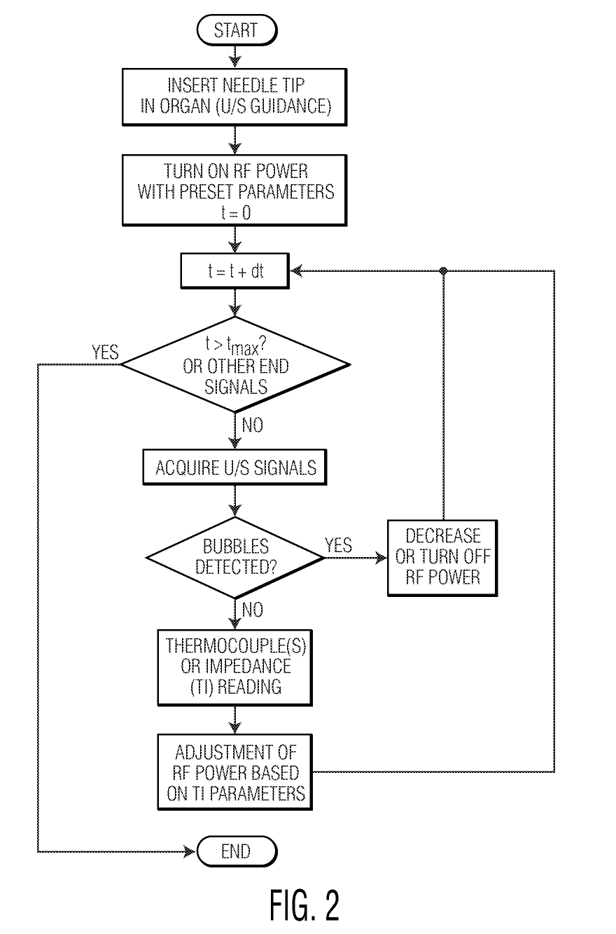

FIG. 2 is a flowchart showing regulation of RF power using feedback received from ultrasound signals.

Ultrasound imaging for interventional guidance of RF ablation therapy has a wide variety of applications, including echocardiography, abdominal and breast imaging, and tumor ablation. An embodiment of the invention is shown in FIG. 1. An ultrasound imaging system, e.g. an ultrasound scanner or ultrasound probe, is used to obtain an ultrasound image of a target area, for example an organ, a tissue, or a tumor. An RF probe, powered by an RF power generator, is inserted into the target area. The positioning of the RF probe can be guided using ultrasound images obtained by the ultrasound imaging system. The ultrasound imaging system also serves as a feedback control mechanism, relaying a feedback signal to the RF power generator, allowing power to the RF probe to be decreased or turned off if bubbles begin to form.

As shown in FIG. 2, a tip of an RF probe is inserted into a target area, e.g. an organ, a tissue, or a tumor, with the guidance of ultrasound to assure proper placement of the RF probe. An RF power generator is turned on with preset parameters, and RF power is generated. The RF power generator operates until an end signal is prompted. For example, if the RF power generator has been operating for an amount of time (t) greater than a maximum amount of time (tₘₐₓ), the RF power generator is automatically turned off. If tₘₐₓ has not been reached, then ultrasound images continue to be acquired. If bubbles are detected using ultrasound images, a feedback signal is generated which, for example, decreases or turns off the RF power. The RF power can be decreased or turned off automatically, or a user can adjust the RF power manually in response to an alert or notification from the system. If no bubbles are detected, then a reading, for example a thermocouple reading or impedance reading, is obtained, and RF power can be adjusted based on the thermocouple or impedance parameters.

An embodiment of the invention includes an ultrasound imaging system, e.g. an ultrasound scanner or ultrasound probe. An ultrasound probe is placed on the body of the patient. An ultrasound imaging system shows an image the organ or tissue of interest through an ultrasound coupling gel. The ultrasound imaging system is used initially to provide a pre-treatment image of a target area, e.g. an organ, tissue, or tumor, which is used for calibration. Continuous real-time acquisition of additional images is maintained by the ultrasound imaging system.

The ultrasound imaging system can also be used for guiding insertion of the RF probe into a target area, such as an organ, tissue, or tumor. The placement of the RF probe into an optimal location, the time of treatment and power deposition should be adequately controlled. Many factors are taken into account when choosing an optimal location for the RF probe. The size and localization of the tumor with respect to other anatomic structures are particularly important. In an exemplary case, the diameter of ablated volume is typically limited to about 2 to about 3 cm; multiple insertions are sometimes required to treat larger tumors. This requires treatment planning, and an imaging modality that allows guidance of needle insertion and that displays the extent of the ablated region.

The RF probe includes a needle portion, which is inserted into the target area, e.g. an organ, a tissue, or a tumor. The RF probe is usually inserted percutaneously, i.e. through the skin. During treatment, an adjuvant saline is infused at the tip of the RF probe. Ground pads are applied on another body surface of the patient, for example the thighs, prior to the RF power generator being turned on.

The RF power generator is turned on, causing heat to be generated in the tissue neighboring the RF probe tip by passage of RF current. RF electrodes are located at the tip of the RF probe, and allow RF power to be generated at the target area. An intraoperative image is produced from the continuous real-time acquisition. The pre-treatment image and the intra-operative image are compared to generate a feedback signal. The feedback signal is relayed to an RF power generator, and RF power is altered in response to the feedback signal to improve quality of the intra-operative image.

The ultrasound imaging system is equipped with a bubble detector, which allows the presence of bubbles to be detected throughout the RF ablation procedure, and produces a feedback signal. The bubble detector compares the pre-treatment image and an intra-operative image. The bubble detector can also include or be associated with, for example, a passive cavitation detector, a microphone, or a stethoscope. The detection scheme of the bubble detector can be based on acquired scattered ultrasound waves, and can also rely on different types of acoustic features, including but not limited to sudden variation of echogenicity (e.g. in the image, or in a region of interest around a RF probe tip), variation in the Doppler spectra in duplex imaging, and non-linear detection schemes developed for microbubble contrast, such as the detection of strong harmonic and/or subharmonic signals.

A comparison between the pre-treatment image and an intra-operative image occurs in response to an index that determines the presence of bubbles in the target area. The index is derived from an ultrasonic image that indicates the presence of bubbles. As bubbles often appear as highly echogenic pockets, a decision can be made on the examination of several ultrasound features. If no bubbles are detected, comparison between the pre-treatment image and an intra-operative image prompts a thermocouple reading or an impedance reading to be obtained.

Detection of the presence of bubbles in the target area initiates an event in a closed loop feedback system. When the index is higher than a certain threshold, a feedback signal is automatically sent to the RF generator. In response, there will be a decrease or a temporary extinction of the RF power generator signal, or an adjustment of power to other sections, tips or prongs of the RF probe. Alternatively, a user can initiate the alterations in RF power in an open loop feedback system.

The feedback generated by the system avoids increased heating and therefore limits boiling. As it is known that cell necrosis is triggered at temperatures lower than the boiling point, and that cell sensitivity to thermal treatments can also be increased by adjuvant therapy, e.g. chemotherapy or saline injection, it is expected that the extent of the coagulated volume should not be reduced even when preventing the occurrence of bubbles in the ultrasound imaging field.

It will furthermore be apparent that other and further forms of the invention, and embodiments other than the specific and exemplary embodiments described above, may be devised without departing from the spirit and scope of the appended claims and their equivalents, and therefore it is intended that the scope of this invention encompasses these equivalents and that the description and claims are intended to be exemplary and should not be construed as further limiting.

What is claimed is:

1. A method for monitoring and regulating radiofrequency (RF) ablation therapy to improve quality of imaging, the method comprising:
imaging a target area using an ultrasound imaging system to provide a pre-treatment image for calibration, and maintaining continuous real-time acquisition of at least one additional image;
inserting an RF probe into the target area and generating an RF current to heat the target area near a tip of the RF probe, and producing at least one intra-operative image from the continuous real-time acquisition; and
comparing the pre-treatment image and the intra-operative image to generate a feedback signal, wherein the feedback signal is relayed to an RF power generator, and altering RF power in response to the feedback signal, to improve quality of the intra-operative image.

2. The method according to claim 1, wherein comparing the pre-treatment image and the intra-operative image further comprises responding to an index that determines a presence in the target area of at least one bubble.

3. The method according to claim 1, wherein the feedback signal comprises a variation in an acoustic feature.

4. The method according to claim 3, wherein the acoustic feature is at least one selected from the group consisting of: a variation in echogenicity, a variation in Doppler spectra in duplex imaging, and a non-linear detection scheme.

5. The method according to claim 4, wherein the non-linear detection scheme comprises harmonic signals and sub-harmonic signals.

6. The method according to claim 1, wherein comparing the pre-treatment image and the intra-operative image further comprises obtaining a thermocouple reading or an impedance reading.

7. A system comprising:
an ultrasound scanner, wherein the ultrasound scanner acquires a pre-treatment image of a target area for calibration, and at least one additional image of the target area;
a radiofrequency (RF) probe, wherein the RF probe is inserted into the target area;
an RF power generator; and
a bubble detector, wherein the bubble detector indicates a presence of at least one bubble in the target area and produces a feedback signal, wherein the RF power generator is altered in response to the feedback signal.

8. The system according to claim 7, wherein the bubble detector further compares the pre-treatment image and at least one intra-operative image.

9. The system according to claim 7, wherein the bubble detector further comprises at least one selected from the group consisting of: a passive cavitation detector, a microphone, and a stethoscope.

10. The system according the claim 7, wherein the bubble detector further determines a variation in echogenicity, a variation in Doppler spectra in duplex imaging, and a non-linear detection scheme.

11. The system according to claim 10, wherein the non-linear detection scheme comprises harmonic signals and sub-harmonic signals.

12. The system according to claim 7, wherein detection of a presence in the target area of at least one bubble initiates at least one event in a closed loop feedback system.

13. The system according to claim 12, wherein the event further comprises an alteration in RF power.

14. The system according to claim 13, wherein the alteration in RF power further comprises an alteration of power to at least one tip of the RF probe.

15. The system according to claim 12, wherein the event further comprises a temporary extinction of an RF power generator signal.

16. The system according to claim 7, wherein a user is notified of a detection of a presence in the target area of at least one bubble, and wherein the user initiates at least one event in an open loop feedback system.

17. The system according to claim 16, wherein the event further comprises an alteration in RF power.

18. The system according to claim 17, wherein the alteration in RF power further comprises a temporary extinction of the RF power generator signal

19. The system according to claim 18, wherein the event further comprises an alteration of power to at least one tip of the RF probe.
